(19) United States
(12) Patent Application Publication
(10) Pub. No.: US 2022/0012884 A1
(43) Pub. Date: Jan. 13, 2022

(54) IMAGE ANALYSIS SYSTEM AND ANALYSIS METHOD

(71) Applicant: NOUL CO., LTD., Yongin-si, Gyeonggi-do (KR)

(72) Inventors: Young Min SHIN, Suji-gu, Yongin-si, Gyeonggi-do (KR); Dong Young LEE, Yongin-si, Gyeonggi-do (KR)

(21) Appl. No.: 17/294,596

(22) PCT Filed: Nov. 19, 2019
(86) PCT No.: PCT/KR2019/015830

§ 371 Date: May 17, 2021

(30) Foreign Application Priority Data
Nov. 19, 2018 (KR) 10-2018-0142831

(51) Int. Cl. G06T 7/00; G06T 7/11

(52) U.S. Cl. CPC G06T 7/0014; G06T 7/11; G06T 2207/30242; G06T 2207/30024; G06T 2207/20081

(57) ABSTRACT

An image analysis method according to one exemplary embodiment of the present disclosure may include: obtaining an unstained cell image; obtaining at least one feature map comprised in the cell image; and identifying a type of cell corresponding to the feature map by using a preset criterion.

Therefore, according to the image analysis method according to one exemplary embodiment of the present disclosure, it is possible to rapidly provide cell image analysis results using an unstained cell image.

### TECHNICAL FIELD

The following exemplary embodiments relate to an image analysis system and an analysis method, and more particularly, a method of identifying a type of cell in an unstained cell image.

### BACKGROUND ART

In general, when cells are analyzed through microscopic images of blood, they undergo staining treatment. This is because various types of cells may be visually distinguished through images due to penetration of pigments into the nuclei and cytoplasm of the cells by staining.

However, blood staining is cumbersome, and visual identification of the type must be performed by an expert. Thus, blood staining is a method requiring much time and high economic costs.

Accordingly, it is necessary to develop an image analysis method capable of automatically identifying cells from unstained blood images.

### DESCRIPTION OF EMBODIMENTS

### Technical Problem

An object of the following exemplary embodiments is to automatically identify types of cells from an unstained blood image.

### Solution to Problem

According to one exemplary embodiment of the present disclosure, provided is an image analysis method, the method including obtaining an unstained cell image; obtaining at least one feature map included in the cell image; and identifying a type of cell corresponding to the feature map by using a preset criterion.

In this regard, the preset criterion may be a criterion which is pre-learned to classify the type of cell included in the unstained cell image.

Further, the preset criterion may be learned using training data obtained by matching label information of a reference image after staining with a target image before staining.

Further, the preset criterion may be continuously updated to accurately identify the type of cell from the unstained cell image.

In this regard, the matching of the label information may include extracting one or more features from the target image and the reference image; matching features of the target image and the reference image; and transmitting label information included in the reference image to a pixel corresponding to the target image.

Further, the method may further include segmenting the unstained cell image, based on a user's region of interest.

Further, it is possible to identify the type of cell according to the preset criterion for each region of the segmented image.

It is also possible to further provide the counted number of each type of the identified cell.

It is also possible to further provide a diagnosis result regarding a specific disease, based on information of the identified cell type.

According to another exemplary embodiment of the present disclosure, provided is a learning method using at least one neural network, the learning method including obtaining one or more training data of unstained blood; generating at least one feature map from the training data; outputting prediction data of the feature map, based on one or more predefined categories; and tuning a parameter applied to the network, based on the prediction data, wherein the above-described steps may be repeatedly performed until preset termination conditions are satisfied.

In this regard, the training data may include label information about one or more cells included in the blood.

The label information may be obtained by matching label information of reference data after staining with unstained target data.

Further, the training data may be data segmented according to the preset criterion.

Further, the training data may be applied as a plurality of segments according to the user's region of interest.

Further, when it is determined that the preset termination conditions are satisfied, the learning may be terminated.

According to still another exemplary embodiment of the present disclosure, provided is a computer-readable medium having recorded thereon a program for executing the above-described methods on a computer.

### Advantageous Effects of Disclosure

According to the following exemplary embodiments, it is possible to rapidly provide a cell image analysis result, because a staining process is omitted.

According to the following exemplary embodiments, it is also possible to provide a high-accuracy cell image analysis result without entirely relying on a medical expert.

Effects by the exemplary embodiments of the present disclosure are not limited to the above-described effects, and effects not mentioned may be clearly understood by those of ordinary skill in the art from the present disclosure and the accompanying drawings.

### BRIEF DESCRIPTION OF DRAWINGS

FIG. 1 is a block diagram illustrating an entire configuration of an image analysis system according to an exemplary embodiment of the present disclosure;

FIG. 2 is a diagram illustrating an operation of an image capture device according to an exemplary embodiment of the present disclosure;

FIG. 3 is a diagram illustrating cell images captured by an image capture device according to an exemplary embodiment of the present disclosure;

FIGS. 4 and 5 are diagrams each illustrating a configuration of a neural network according to an exemplary embodiment of the present disclosure;

FIG. 6 is a block diagram illustrating a configuration of an image analysis module according to an exemplary embodiment of the present disclosure;

FIG. 7 is a diagram illustrating an operation performed in an image analysis module according to an exemplary embodiment of the present disclosure;

FIG. 8 is a flowchart illustrating an image analysis method according to a first exemplary embodiment of the present disclosure;

FIG. 9 is a flowchart illustrating an image analysis method according to a second exemplary embodiment of the present disclosure;

FIG. 10 is a flowchart illustrating a learning method according to a third exemplary embodiment of the present disclosure; and

FIG. 11 is a diagram illustrating an image synthesis method for converting an unstained blood cell image into a stained blood cell image according to a fourth exemplary embodiment of the present disclosure.

### MODE OF DISCLOSURE

The above-described objects, features, and advantages of the present disclosure will become more apparent from the following detailed description when taken in conjunction with the accompanying drawings. Although the present disclosure may be variously modified and may have several exemplary embodiments, specific exemplary embodiments will be illustrated in drawings and will be explained in detail.

In the drawings, the thicknesses of layers and regions are exaggerated for clarity. When an element or a layer is referred to as being “on” or “above” another element or layer, it means that each layer or element is directly formed on another layer or element, or other layers or elements may be formed therebetween. The same reference numerals will be used throughout to designate the same components. Also, elements having the same function within a scope of the same concept illustrated in drawings of respective embodiments will be described by using the same reference numerals.

Detailed descriptions of known functions or configurations related to the present disclosure will be omitted when they would unnecessarily obscure the subject matter of the present disclosure. Further, numerals (e.g., first, second, etc.) used to describe the present disclosure are merely identifiers for discriminating one component from other components.

The suffixes “module” and “unit” for components used in the description below are assigned or mixed in consideration of easiness in writing the specification and do not have distinctive meanings or roles by themselves.

According to one aspect of the present disclosure, provided is an image analysis method, the method including obtaining an unstained cell image; obtaining at least one feature map included in the cell image; and identifying a type of cell corresponding to the feature map by using a preset criterion.

In this regard, the preset criterion may be a criterion which is pre-learned to classify the type of cell included in the unstained cell image.

Further, the preset criterion may be learned using training data obtained by matching label information of a reference image after staining with a target image before staining.

Further, the preset criterion may be continuously updated to accurately identify the type of cell from the unstained cell image.

In this regard, the matching of the label information may include extracting one or more features from the target image and the reference image; matching features of the target image and the reference image; and transmitting label information included in the reference image to a pixel corresponding to the target image.

Further, the image analysis method according to an aspect of the present disclosure may further include segmenting the unstained cell image, based on an user's region of interest.

In this regard, it is possible to identify the type of cell according to the preset criterion for each region of the segmented image.

It is also possible to further provide the counted number of each type of the identified cell.

It is also possible to further provide a diagnosis result regarding a specific disease, based on information of the identified cell type.

According to another aspect of the present disclosure, provided is a learning method for analyzing a blood image using at least one network, the learning method including obtaining one or more training data of unstained blood; generating at least one feature map from the training data; outputting prediction data of the feature map, based on one or more predefined categories; and tuning a parameter applied to the network, based on the prediction data, wherein the above-described steps may be repeatedly performed until preset termination conditions are satisfied.

In this regard, the input data may include label information about one or more cells included in the blood.

Further, the label information may be obtained by matching label information of reference data after staining with unstained target data.

Further, the training data may be data segmented according to the preset criterion.

Further, the training data may be applied as a plurality of segments according to the user's region of interest.

Further, when it is determined that the preset termination conditions are satisfied, the learning may be terminated.

According to still another aspect of the present disclosure, provided is a computer-readable medium having recorded thereon a program for executing the above-described methods on a computer.

Hereinafter, a blood test method using an unstained blood image will be introduced and described.

### 1. Blood Cell Analysis Method

A general blood test (CBC, Complete Blood Cell Count) is one of the most basic tests performed for diagnosis, treatment, and follow-up of diseases. Through this test, various indicators regarding blood cells, e.g., red blood cells, white blood cells, and platelets, and bacteria, etc., present in the blood may be identified.

Blood test methods include a method of measuring the number of cells using an automated analyzer, and a method of directly observing the number and morphological abnormalities of blood cells by an expert.

When the automated analyzer is used, it provides fast and reliable results for the number and size of cells, and changes in the size of cells, but there is a limitation in that it is difficult to identify a specific shape.

In contrast, the direct observation by an expert may precisely observe the number and morphological abnormalities of blood cells through a microscope.

Representatively, a peripheral blood smear test is a test, in which peripheral blood is collected, smeared on a slide glass, and then stained, followed by observing blood cells, bacteria, parasites, etc. in the stained blood.

Here, red blood cells may be used in diagnosing anemia and parasites such as malaria present in red blood cells. Further, white blood cells may be used in determining myelodysplastic syndrome, leukemia, causes of infection and inflammation, megaloblastic anemia, etc. Further, platelets may help identify a myeloproliferative disorder, platelet satellitism, etc.

In general, the peripheral blood smear test may include a process of smearing blood, a process of staining the smeared blood, and a process of observing the stained blood.

The process of smearing blood is a process of spreading blood on a plate such as a slide glass. For example, after dropping a blood drop on a plate, blood may be spread on the plate using a member for smearing.

The process of staining blood is a process of infiltrating a staining sample into the nuclei and cytoplasm of cells.

Here, as a staining sample for nuclei, a basic staining sample, e.g., methylene blue, toluidine blue, hematoxylin, etc. may be mainly used. In addition, as a staining sample for cytoplasm, an acidic staining sample, e.g., eosin, acid fuchsin, orange G, etc. may be used.

In addition, the blood staining method may be performed in various ways depending on the purpose of the test. For example, Romanowsky staining, such as Giemsa staining, wright staining, Giemsa-Wright staining, etc. may be used.

Alternatively, for example, simple staining, Gram staining, etc., accompanied by a bacterial test, may be used.

Therefore, the medical technician may visually distinguish the types of cells by observing the image of the stained cells through an optical device.

However, most of the blood test processes as described above is performed manually by an expert, and therefore, various methods have been developed to perform the blood test more quickly and conveniently.

For one example, a blood test method using a blood staining patch is a method of more simply performing staining by bringing a patch containing a staining sample into contact with blood smeared on a plate.

Here, the patch may store one or more staining samples, and may transfer the staining samples to blood smeared on the slide glass. In other words, when the smeared blood and the patch are brought into contact, the staining sample in the patch moves to the blood, thereby staining the cytoplasm or nuclei in the blood.

For another example, there is a method of identifying the type of cells by capturing an image of the entire surface of the plate, on which the stained blood is smeared, using an optical device, and then analyzing the image of the stained blood using various image processing techniques.

However, both methods still employ the blood staining process, resulting in loss of time. Therefore, to provide a faster blood analysis result, an image analysis system capable of automatically identifying the types of cells from an unstained blood image is needed.

Hereinafter, a blood test performed by blood smear without involving the staining process will be introduced and described.

### 2. Image Analysis System

An image analysis system according to one exemplary embodiment of the present disclosure is a system for automatically identifying the type of cell using an unstained blood image.

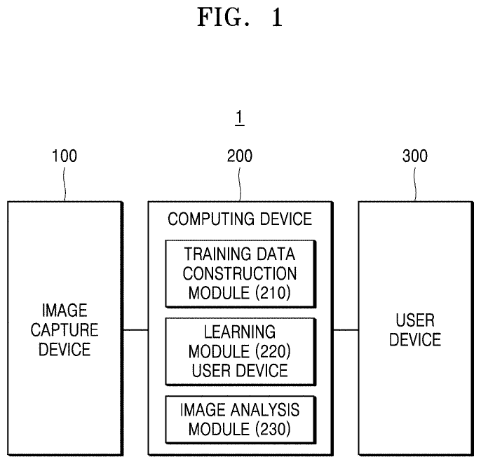

FIG. 1 is a block diagram illustrating an entire configuration of the image analysis system according to one exemplary embodiment of the present disclosure.

The image analysis system 1 according to one exemplary embodiment of the present disclosure may include an image capture device 100, a computing device 200, a user device 300, etc.

In this regard, the image capture device 100, the computing device 200, and the user device 300 may be connected to each other by a wired or wireless communication, and various types of data may be transmitted and received between respective components.

In addition, as shown in FIG. 1, the computing device 200 may include a training data construction module 210, a learning module 220, an image analysis module 230, etc.

In the image analysis system 1 according to one embodiment of the present disclosure, only the case where all of the above-described modules are placed in one computing device 200 is exemplified, but the training data construction module 210, the learning module 220, and the image analysis module 230 may be provided through separate devices, respectively.

Alternatively, one or more functions of the training data construction module 210, the learning module 220, and the image analysis module 230 may be integrated to be provided as one module.

Hereinafter, for the convenience of description, the functions of the above-described modules that are separately provided in one computing device 200 will be introduced and described.

Meanwhile, although not shown in the drawings, the computing device 200 may further include one or more processors, memories, etc. to perform a variety of image processing and image analysis.

Hereinafter, operations performed by respective components will be described in detail.

2.1 Blood Image Capture

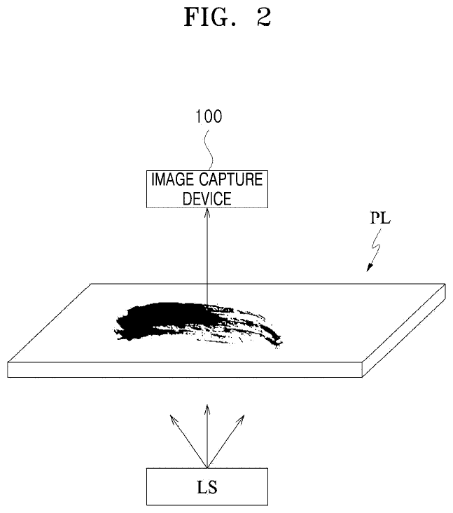

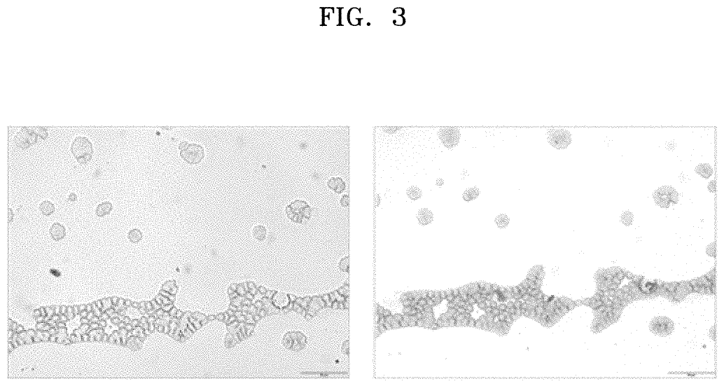

Hereinafter, a process of obtaining a blood image through the image capture device according to one embodiment of the present disclosure will be described with reference to FIGS. 2 and 3.

FIG. 2 is a diagram illustrating an operation of the image capture device according to one exemplary embodiment of the present disclosure. In addition, FIG. 3 is a diagram illustrating cell images captured by the image capture device according to one exemplary embodiment of the present disclosure.

The image capture device 100 may be an optical device for obtaining an image of blood.

The optical device 100 may be various types of imaging devices capable of obtaining an image of blood for detecting blood cells, bacteria, etc. in the blood within a range that does not damage cells.

In this regard, the blood image may be obtained in various ways by adjusting direction of a light source, imaging at different wavelength bands, adjusting the focus, adjusting the aperture, etc.

For example, the optical device 100 may include an optical sensor consisting of CCD, CMOS, etc., a barrel providing an optical path, a lens for adjusting magnification and focal length, a memory for storing an image captured from the optical sensor, etc.

For example, as shown in FIG. 2, the image capture device 100 may be disposed on the surface of the slide glass (PL) on which blood is smeared. In this regard, the light source (LS) may be disposed on the rear surface of the slide glass (PL). In this case, the image capture device 100 may receive light which is irradiated from the light source (LS) and passes through the slide glass (PL), and may capture an image of blood smeared on the slice glass (PL).

Accordingly, referring to FIG. 3, a blood image before staining (left) and a blood image after staining (right) may be obtained using the image capture device 100.

2.2 Training Data Construction

To learn a classification criterion for identifying the cell type from the unstained blood image, label information about the cells in the unstained blood image is required.

Therefore, it is necessary to construct training data regarding unstained blood images using label information about stained blood images which are read by experts.

Hereinafter, an operation performed in the training data construction module that generates training data for use in learning the cell classification criterion will be described.

The training data construction module 210 is a configuration for constructing training data which may be used in learning for image analysis in the learning module 220 described below.

In other words, the training data generated by the training data construction module 210 may be an unstained blood image, and the training data may include label information about one or more cells included in the blood image.

The label information may include, for example, species type, location information, or area information of cells included in the blood image.

Hereinafter, a process of generating the training data by the training data construction module 210 will be described in detail.

First, images of a slide of blood before staining and a slide of blood after staining may be captured using the above-described image capture device 100.

The training data construction module 210 may obtain at least one pair of images photographing slides of blood before and after staining from the image capture device 100, and may generate training data by using the one pair of images as input data.

For example, the training data generated by the training data construction module 210 may be obtained by matching label information of a reference image after staining with a target image before staining.

In this regard, the label information of the reference image after staining may be input by an experienced technician.

Further, various image processing algorithms may be applied to transfer label information of the reference image to the target image. For example, an image registration algorithm may be applied.

Image registration is a process of transforming different sets of data into a single coordinate system. Therefore, image registration involves spatially transforming the source image to align with the target image.

The different sets of data may be obtained from, for example, different sensors, times, depths, or viewpoints.

The image registration method may be classified into an intensity-based method and a feature-based method.

The intensity-based method is a method of comparing intensity patterns in images via correlation metrics.

The intensity-based method registers entire images or sub-images. When sub-images are registered, centers of corresponding sub-images are treated as corresponding features.

The feature-based method finds correspondence between features in images, such as points, lines, and contours.

The feature-based method establishes a correspondence between distinct points in images. Knowing the correspondence between points in images, a geometrical transformation is then determined to map the target image to the reference images, thereby establishing point-by-point correspondence between the reference and target images.

In this regard, registration of images may be performed by various methods, such as manual, interaction, semi-automatic, automatic methods, etc.

The above-described registration of different images is a field that has been studied for a very long time in the field of computer vision, and the feature-based registration method has shown good results for various types of images.

Hereinafter, transmitting of label information of a reference image to a target image using a feature-based image registration algorithm will be exemplified and described.

First, features may be extracted from the input image using a detector such as scale invariant feature transform (SIFT), speeded up robust features (SURF), features form accelerated segment test (FAST), binary robust independent elementary features (BRIEF), oriented fast and rotated brief (ORB), etc.

Next, it is possible to determine an optimal motion while removing outlier matching between the extracted features. For example, an algorithm such as random sample consensus (RANSAC) may be used.

Here, motion may be regarded as a transformation function that provides correspondences between pixels included in two images, and through this, label information of one image may be transferred to another image.

Accordingly, after the registration process between two images or a pair of images is completed, the label information included in the stained reference image may be transferred to the unstained target image.

In other words, the training data construction module 210 may perform image registration using, as input data, a plurality of sets of blood image data before and after staining which are obtained from the image capture device 100, and thus unstained training data including label information may be constructed.

Meanwhile, the training data may be stored in a storage unit (not shown) placed in the training data construction module 210 or a memory (not shown) of the computing device 200, and may be used to perform image data learning and evaluation of the learning module 220 described below.

2.3 Classification Criterion Learning

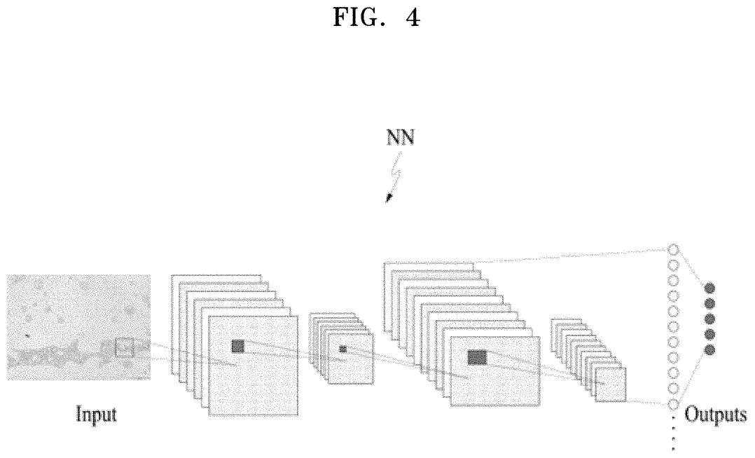

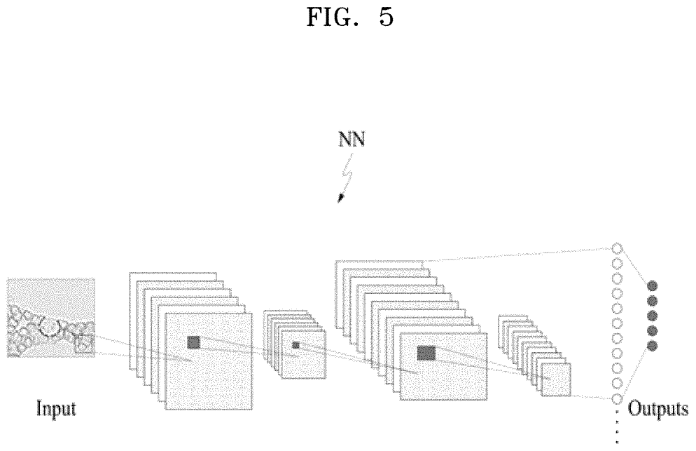

Hereinafter, an operation performed in a learning module that performs learning using a plurality of training data will be described with reference to FIGS. 4 and 5.

The learning module 220 is a configuration for learning a classification criterion for identifying the types of cells included in the blood image by using the training data regarding the unstained blood images generated by the training data construction module 210 described above.

As described above, the plurality of training data may be unstained blood images including label information about each cell type.

In addition, a category for one or more types of cells included in the blood image may be predefined by a user.

For example, in the case of learning the classification criterion for classifying the species of white blood cells, the user may categorize the species of white blood cells, such as neutrophils, eosinophils, basophils, lymphocytes, monocytes, etc.

In other words, the user may categorize training data according to the type of cell to be classified, and the learning module 220 may learn a classification criterion for distinguishing the type of cell using the categorized training data. For example, the categorized training data may be data pre-segmented for each cell type.

Meanwhile, as shown in FIG. 1, the learning module 220 may be provided as a part of the computing device 200 for performing image analysis. In this regard, one or more machine learning algorithms for performing machine learning may be provided in the learning module 220.

Specifically, various machine learning models may be used in the learning process according to one exemplary embodiment of the present disclosure. For example, a deep learning model may be used.

Deep learning is a set of algorithms that attempt a high level of abstraction through a combination of several nonlinear transformation methods. As a core model of deep learning, a deep neural network (DNN) may be used. The deep neural network (DNN) includes several hidden layers between an input layer and an output layer, and deep belief network (DGN), deep auto encoders, convolutional neural network (CNN), recurrent neural network (RNN), generative adversarial Network (GAN), etc. may be used depending on a learning method or structure.

Here, learning is to understand the characteristics of data according to a given purpose, and in deep learning, connection weights are adjusted.

For example, the convolutional neural network (CNN), which may be applied to learning two-dimensional data such as images, may be composed of one or several convolution layers and a pooling layer, and fully connected layers, and may be trained through a backpropagation algorithm.

For example, the learning module 220 may obtain one or more feature maps from unstained training data using one or more convolutional neural networks (CNNs), and may learn a classification criterion for distinguishing one or more cells included in the unstained training data according to a predefined category using the feature maps.

In this regard, the learning module 220 may perform learning using various types of convolutional neural networks (CNN) suitable for classifying cells included in the blood image, such as a deep learning architecture, e.g., LeNet, AlexNet, ZFNet, GoogLeNet, VggNet, ResNet, etc., or a combination thereof, etc.

Hereinafter, learning performed using one or more neural networks will be exemplified and described with reference to FIGS. 4 and 5.

Here, the neural network may be composed of a plurality of layers, and the layer configuration may be changed, added, or removed according to a result of learning.

FIGS. 4 and 5 are diagrams, each illustrating a configuration of the neural network according to an exemplary embodiment of the present disclosure.

As shown in FIGS. 4 and 5, the neural network may be a convolutional neural network, and one or more training data may be applied as input data of the neural network.

In this regard, the input data (Input) may be all image data obtained from the image capture device 100 as shown in FIG. 4. Alternatively, as shown in FIG. 5, the input data may be data segmented according to a preset criterion.

For example, the learning module 220 may segment one or more training data into a preset size. Alternatively, for example, the learning module 220 may segment training data according to an user's region of interest (ROI).

In addition, the input data may be data obtained by processing unstained blood image data through pre-processing.

The image pre-processing is for processing an image so that the computer is allowed to easily recognize the image, and may include, for example, brightness transformation and geometric transformation of image pixels, etc.

For example, the input data may be those obtained by converting the blood image data into a binary image through pre-processing.

For another example, the input data may be those obtained by removing an erroneous feature included in the image through pre-processing.

Meanwhile, various image processing algorithms may be applied to the image pre-processing, and the speed and/or performance of learning may be improved by performing the image pre-processing before inputting the blood image to the neural network.

In addition, referring to FIGS. 4 and 5, the neural network may include a plurality of layers, and the plurality of layers may include one or more of a convolution layer, a pooling layer, and a fully connected layer.

In this regard, the neural network may consist of a process of extracting features in the blood image and a process of classifying the image.

For example, feature extraction of an image may be performed by extracting a plurality of features included in the unstained blood image through a plurality of convolutional layers, and generating at least one feature map (FM) using the plurality of features. In other words, the learning module 220 may generate at least one feature map using a plurality of layers of the neural network.

The features may include, for example, edge, sharpness, depth, brightness, contrast, blur, shape, or combination of shapes, etc., and the features are not limited to the above-described examples.

The feature map may be a combination of the plurality of features. The user's ROI in the blood image may be identified through at least one feature map.

The ROI may be various cell regions in blood, which are predetermined by the user. For example, the ROI may be neutrophils, eosinophils, basophils, lymphocytes, monocytes, etc. of white blood cells in the blood image.

In addition, classification of the feature map may be, for example, performed by calculating at least one feature map generated through the plurality of layers as a score or probability for one or more predefined categories.

Accordingly, the learning module 220 may learn a classification criterion for identifying the cell type, based on the class score or probability value for the one or more categories.

In this regard, the learning module 220 may tune parameters applied to the neural network by repeatedly performing a learning process until preset termination conditions are satisfied.

In this regard, the learning module 220 may tune parameters for the plurality of layers of the neural network, for example, in a manner that propagates an error of a result of learning the neural network using a backpropagation algorithm.

In addition, the user may set, for example, to repeat the learning process until the loss function of the neural network does not decrease.

Here, the loss function may mean a degree of similarity between correct answer data for input data and output data of the neural network. The loss function is used to guide the learning process of the neural network. For example, a mean square error (MSE), a cross entropy error (CEE), etc. may be used.

Alternatively, the user may set, for example, to repeat the learning process for a predetermined number of times.

Therefore, the learning module 220 may provide the image analysis module 230 described below with optimal parameters for identifying cells in the blood image.

A learning process performed by the learning module 300 will be described in detail with reference to the related exemplary embodiments below.

Meanwhile, the learning module 220 may further evaluate accuracy and error of learning by using data which are not used in learning among a plurality of training data obtained from the above-described training data construction module 210.

For example, the learning module 220 may further increase accuracy of learning by performing evaluation on the network at predetermined intervals.

2.4 Image Prediction

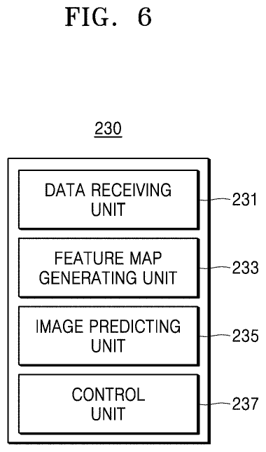

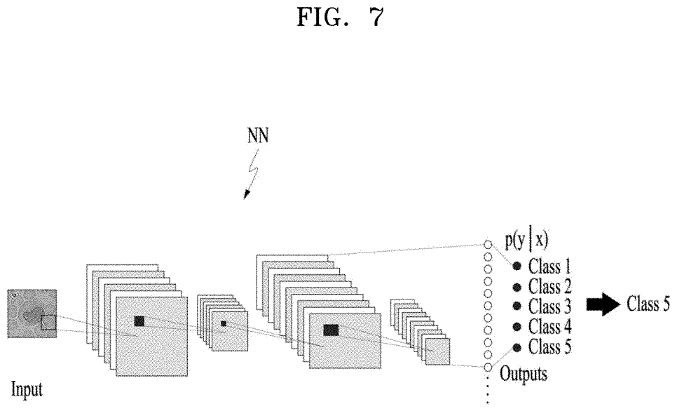

Hereinafter, operations performed by the image analysis module for predicting cell types included in a blood image using pre-learned classification criteria will be described with reference to FIGS. 6 and 7.

FIG. 6 is a block diagram illustrating a configuration of the image analysis module according to one exemplary embodiment of the present disclosure. In addition, FIG. 7 is a diagram illustrating an operation performed in the image analysis module according to one exemplary embodiment of the present disclosure.

The image analysis module 230 is a component for analyzing the blood image obtained from the image capture device 100 using a pre-learned classification criterion.

The pre-learned classification criterion may be an optimal parameter value transmitted from the above-described learning module 220.

In addition, the image analysis module 230 may be provided as a part of the computing device 200 as described above. Alternatively, the image analysis module may be provided in a separate computing device which is separate from the above-described learning module 220.

For example, the computing device may include at least one processor, memory, etc. One or more image processing algorithms, machine learning algorithms, etc. may be provided in the at least one processor.

Alternatively, the image analysis module 200 may be, for example, provided in the form of a software program executable on a computer. The program may be previously stored in the memory.

Referring to FIG. 6, the image analysis module 230 may include a data receiving unit 231, a feature map generating unit 233, an image predicting unit 235, a control unit 237, etc.

The data receiving unit 231 may receive one or more image data captured from the above-described image capture device 100. The image data may be an unstained blood image, and may be obtained in real time from the image capture device 100.

Alternatively, the data receiving unit 231 may receive one or more image data previously stored in the user device 300 described below. The image data may be an unstained blood image.

The feature map generating unit 233 may generate one or more feature maps by extracting features in the input image.

The input image may be an image which is sampled, based on the user' preset ROI. Alternatively, the input image may be an image segmented according to the preset criterion.

For example, the feature map generating unit 233 may extract one or more features included in the input image using a neural network (NN) which is optimized through the above-described learning module 220, and may generate at least one feature map by combining the features.

The image predicting unit 235 may predict the types of cells included in the input image according to the classification criterion learned from the above-described learning module 220.

For example, the image predicting unit 235 may classify the input image into one of defined categories according to the pre-learned criterion using the one or more feature maps.

Referring to FIG. 7, the blood image obtained by segmenting the blood image captured from the image capture device 100 according to the preset criterion may be input to NN. In this regard, NN may extract features in the blood image through a plurality of layers, and may generate one or more feature maps using the features.

The feature map may be predicted to correspond to class 5, which is one of predefined categories, class 1, class 2, class 3, class 4, and class 5 according to the criterion pre-learned through the above-described learning module 220. For example, at least one feature map obtained from the image which is input to the neural network shown in FIG. 7 may be predicted to correspond to monocytes among the types of white blood cells.

The control unit 240 may be a component for directing an image prediction operation which is performed by the image analysis module 230.

For example, the control unit 240 may obtain a parameter that is updated according to the learning result by the above-described learning module 220, and the parameter may be transferred to the feature map generating unit 233 and/or the image predicting unit 235.

A method of identifying cells in the blood image, which is performed by the image analysis module 200, will be described in detail with reference to the related exemplary embodiment below.

2.5 Image Analysis

Hereinafter, utilization of the result of the blood image analysis performed by the above-described image analysis module 200 will be exemplified and described.

A user device 400 may obtain the image analysis result from the above-described image analysis module 300.

In this regard, various information related to the blood image obtained from the image analysis module 300 may be displayed through the user device 400. For example, the user device 400 may include information regarding the number of blood cells according to each type and the number of bacteria.

In addition, the user device 400 may be a device for further providing results of various analyses, such as a blood test, etc., using various information related to the blood image which is obtained from the image analysis module 300.

For example, the user device 300 may be a computer, a portable terminal, etc. of a medical expert or technician. In this regard, the user device 300 may have programs and applications which are installed to further provide various analysis results.

For example, in a blood test, the user device 400 may obtain a result of identifying blood cells, bacteria, etc. in the blood image from the above-described image analysis module 300. In this regard, the user device 400 may further provide information regarding abnormal blood cells, diagnosis results of various diseases, etc. by using a pre-stored blood test program.

Meanwhile, the user device 400 and the above-described image analysis module 300 may be implemented in a single device.

### 3. First Exemplary Embodiment

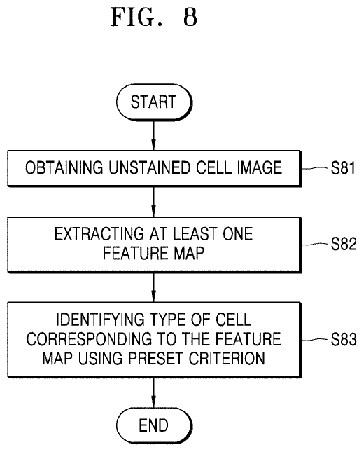

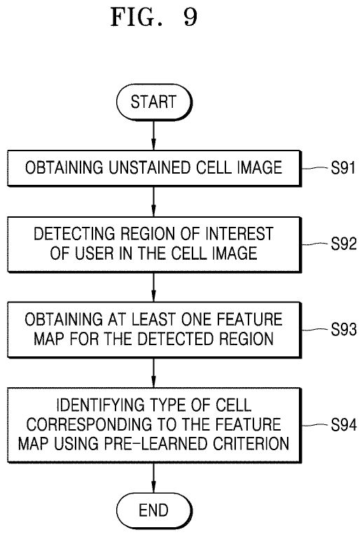

Hereinafter, an image analysis method according to a first exemplary embodiment of the present disclosure will be described with reference to FIGS. 8 and 9.

Hereinafter, in the image analysis system 1 according to the first exemplary embodiment of the present disclosure, use of one or more neural networks to identify one or more types of cells from unstained blood image data will be exemplified and described.

For example, one or more neural networks may be the above-described convolutional neural network (CNN).

For example, the image analysis method according to the first exemplary embodiment of the present disclosure may be to identify a species of white blood cells which are observed from blood image data.

Here, the species of white blood cells may be classified into at least two or more.

For example, the types of white blood cells may include neutrophils, eosinophils, basophils, lymphocytes, monocytes, etc.

FIG. 8 is a flowchart illustrating the image analysis method according to the first exemplary embodiment of the present disclosure.

Referring to FIG. 8, the image analysis method according to the first exemplary embodiment of the present disclosure may include obtaining an unstained cell image S81, obtaining at least one feature map from the cell image S82, and identifying the cell species corresponding to the feature map using the pre-learned criterion S83. The above steps may be performed by the control unit 237 of the above-described image analysis module 230, and each step will be described in detail below.

The control unit 237 may obtain an unstained cell image S81.

For example, the control unit 237 may obtain the unstained cell image from the image capture device 100 in real time.

As described above, the image capture device 100 may obtain an image of blood smeared on a slide glass (PL) in various ways, and the control unit 237 may obtain one or more cell images which are captured from the image capture device 100.

For another example, the control unit 237 may receive one or more pre-stored image data from the user device 300.

For example, the user may select at least one image data, as needed, among a plurality of cell images which are captured by the image capture device 100. In this regard, the control unit 237 may perform the next step by using at least one image data selected by the user.

Alternatively, for example, the control unit 237 may segment the cell image according to a preset criterion, and may perform the next step using one or more segmented image data.

In addition, the control unit 237 may extract at least one feature map from the cell image S82.

In other words, as described above, the feature map generating unit 233 may generate one or more feature maps by extracting features in the cell image obtained from the image capture device 100.

In this regard, the feature map generating unit 233 may extract one or more features included in the input cell image using a neural network (NN) pre-learned through the learning module 220, and may generate one or more feature maps by combining the features.

For example, the one or more feature maps may be generated by a combination of one or more of edge, sharpness, depth, brightness, contrast, blur, and shape in the cell image which is input in S81.

In addition, the control unit 237 may identify the type of cell corresponding to the feature map using the preset criterion S83.

For example, the above-described image predicting unit 235 may predict the types of cells included in the cell image according to the classification criterion pre-learned from the learning module 220.

In other words, the image predicting unit 235 may classify the feature map generated in S82 into one of predefined categories according to the pre-learned classification criterion.

The pre-learned classification criterion may be a pre-learned criterion to classify the types of cells included in the unstained cell image. For example, the pre-learned criterion may be a parameter applied to a plurality of layers included in the neural network (NN).

Also, the predefined category may be predefined by the user. For example, the user may categorize training data according to each type to be classified. In the training data construction module 210, training data may be stored according to each category.

For example, as described above with reference to FIG. 7, the image predicting unit 235 may calculate a score or probability according to each predefined category with respect to at least one feature map generated in S82, and based on this, it is possible to predict which of the predefined categories the feature map will correspond to.

For example, the image predicting unit 235 may calculate a probability of 0.01 for class 1, a probability of 0.02 for class 2, a probability of 0.04 for class 3, a probability of 0.03 for class 4, and a probability of 0.9 for class 5, with respect to the feature map generated in S82. In this regard, the image predicting unit 235 may determine the classification of the feature map as class 5 having 0.9 or more.

In other words, the image predicting unit 235 may classify the feature map into a category having a preset value or more, based on the score or probability for the predefined category of the feature map.

Accordingly, the image predicting unit 235 may predict that the feature map generated in S82 corresponds to class 5 among class 1 to class 5, as described above with reference to FIG. 7.

Meanwhile, the learning module 220 may continuously update and provide the preset criterion to more accurately identify the cell type from the unstained cell image.

### 4. Second Exemplary Embodiment

FIG. 9 is a flowchart illustrating an image analysis method according to a second exemplary embodiment of the present disclosure.

Hereinafter, in the image analysis system 1 according to one exemplary embodiment of the present disclosure, use of one or more neural networks to identify one or more types of cells from unstained blood image data will be exemplified and described.

For example, one or more neural networks may be the above-described convolutional neural network (CNN).

Referring to FIG. 9, the image analysis method according to the second exemplary embodiment of the present disclosure may include obtaining an unstained cell image S91, detecting an user's region of interest in the cell image S92, obtaining at least one feature map from the image related to the detected region S93, and identifying the cell species corresponding to the feature map using the pre-learned criterion S94. The above steps may be performed by the control unit 237 of the above-described image analysis module 230, and each step will be described in detail below.

Unlike the above-described image analysis method according to the first exemplary embodiment, in which the blood image is segmented according to the preset criterion and applied as an input value to the neural network, the image analysis method according to the second exemplary embodiment of the present disclosure may be performed in such a manner that unsegmented image data is applied as an input value to the neural network.

In other words, the image analysis method according to the second exemplary embodiment of the present disclosure may further include detecting a plurality of objects included in the blood image to identify the plurality of objects included in the blood image according to a predefined category. Hereinafter, each of the steps performed by the control unit 237 will be described in order.

The control unit 237 may obtain an unstained cell image S91.

For example, the control unit 237 may obtain the unstained cell image from the image capture device 100 in real time.

As described above, the image capture device 100 may obtain an image of blood smeared on a slide glass (PL) in various ways, and the control unit 237 may obtain one or more cell images which are captured from the image capture device 100.

For another example, the control unit 237 may receive one or more pre-stored image data from the user device 300.

In addition, the control unit 237 may detect one or more user's regions of interest through detection of objects in the cell image S92.

The control unit 237 may apply the unstained cell image as input data to the above-described neural network.

In this regard, the control unit 237 may extract one or more user's ROIs included in the input data using at least one of a plurality of layers included in the neural network.

For example, the ROI may be one or more of neutrophils, eosinophils, basophils, lymphocytes, and monocytes of white blood cells in the blood image. In this regard, the control unit 237 may detect one or more regions of eosinophils, basophils, lymphocytes, and monocytes present in the blood image, and may generate sample image data regarding the detected regions.

Accordingly, the control unit 237 may perform the next step using one or more sample image data regarding one or more ROIs.

In addition, the control unit 237 may extract at least one feature map from the cell image S93.

In other words, as described above, the feature map generating unit 233 may generate one or more feature maps by extracting features in the cell image obtained from the image capture device 100.

In this regard, the feature map generating unit 233 may extract one or more features included in the input cell image using the neural network (NN) pre-learned through the learning module 220, and may generate one or more feature maps by combining the features.

For example, the one or more feature maps may be generated by combination of one or more of edge, sharpness, depth, brightness, contrast, blur, and shape in the cell image input in S81.

In addition, the control unit 237 may identify the cell type corresponding to the feature map using the preset criterion S94.

For example, the above-described image predicting unit 235 may predict the types of cells included in the cell image according to the classification criterion pre-learned from the learning module 220. In other words, the image predicting unit 235 may classify one or more ROIs included in the cell image obtained in S92 into one of predefined categories according to the pre-learned classification criterion.

The pre-learned classification criterion may be a pre-learned criterion to classify the types of cells included in the unstained cell image. For example, the pre-learned criterion may be a parameter applied to a plurality of layers included in the neural network (NN).

Also, the predefined category may be predefined by a user. For example, the user may categorize training data according to a type to be classified, and training data may be stored according to each category in the training data construction module 210.

In addition, the method of classifying the feature map into the predefined categories in the image predicting unit 235 is the same as the image prediction method which has been described above with reference to FIG. 8, and therefore, a detailed description thereof will be omitted.

Meanwhile, the learning module 220 may continuously update and provide the preset criterion to more accurately identify the type of cell from the unstained cell image.

### 5. Third Exemplary Embodiment

Hereinafter, in the above-described image analysis method, a learning method of providing pre-learned optimal parameters for the image analysis module 230 will be described in detail.

Hereinafter, in the image analysis system 1 according to one exemplary embodiment of the present disclosure, use of one or more neural networks to identify one or more types of cells from unstained blood image data will be exemplified and described.

In this regard, the one or more neural networks may be the above-described convolutional neural network (CNN).

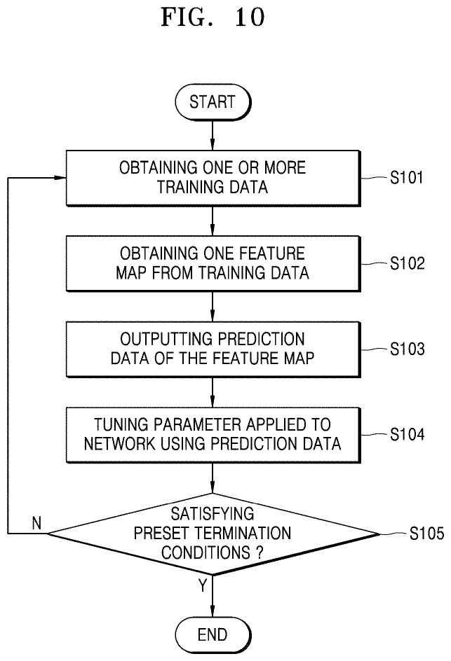

FIG. 10 is a flowchart illustrating a learning method according to a third exemplary embodiment of the present disclosure.

Referring to FIG. 10, regarding to the learning method according to the third exemplary embodiment of the present disclosure, the learning method using at least one neural network may include obtaining one or more training data obtained by registering a target image to label information of a reference image S91, generating at least one feature map from the training data S92, outputting prediction data for the feature map S93, tuning a parameter applied to the network using the prediction data S94, and determining whether the preset termination conditions are satisfied S95.

Hereinafter, the above-described steps performed using the above-described neural network in the above-described learning module 220 will be described with reference to FIGS. 4 and 5.

The learning module 220 may obtain one or more training data.

For example, the learning module 220 may obtain a plurality of training data from the above-described training data construction module 210.

Here, the one or more training data may be an unstained blood image, and may be data including label information regarding the types of cells in the blood image.

As described above, to learn the classification criterion for identifying the types of cells from the unstained blood image, the learning module 220 may use training data previously constructed using a pair of blood images before and after staining.

In addition, the training data may be pre-categorized according to the type of cell by the user. In other words, the user may read the stained blood image data obtained from the image capture device 100 to classify and store the training data according to the type of cell. Alternatively, the user may segment blood image data according to the type of cell to store them in a storage unit which is placed inside the training data construction module 210 or the learning module 220.

In addition, the training data may be data processed through pre-processing. Since various pre-processing methods have been described above, detailed descriptions thereof will be omitted below.

In addition, the learning module 220 may generate at least one feature map from the training data S92.

In other words, the learning module 220 may extract features in the training data using a plurality of layers included in at least one neural network. In this regard, the learning module 220 may generate at least one feature map using the extracted features.

The features may include, for example, edge, sharpness, depth, brightness, contrast, blur, shape, or combination of shapes, etc. The features are not limited to the above-described examples.

The feature map may be a combination of the plurality of features, and the user's ROI in the blood image may be identified through at least one feature map.

The ROI may be various cell regions in blood, which are predetermined by the user. For example, the ROI may be neutrophils, eosinophils, basophils, lymphocytes, monocytes, etc. of white blood cells in the blood image.

In addition, the learning module 220 may output prediction data regarding the feature map S93.

In other words, the learning module 220 may generate at least one feature map through the above-described neural network, and may output prediction data regarding the feature map as a result value through the last layer of the neural network.

The prediction data may be output data of the neural network obtained by calculating similarity between at least one feature map generated in S92 and each of one or more categories pre-defined by the user as a score or a probability having a value between 0 and 1.

For example, with respect to at least one feature map generated in S92, a probability of 0.32 for class 1, a probability of 0.18 for class 2, a probability of 0.40 for class 3, a probability of 0.08 for class 4, and a probability of 0.02 for class 5 may be calculated and stored as a result value.

In this regard, the prediction data may be stored in a memory (not shown) placed in the learning module 220.

In addition, the learning module 220 may tune a parameter applied to the network using the prediction data S94.

In other words, the learning module 220 may reduce errors of the neural network by backpropagating the errors of the result of training the neural network, based on the prediction data output in S92.

Error backpropagation is a method of updating the weights of layers in proportion to an error caused by a difference in output data of the neural network and correct answer data for input data.

Accordingly, the learning module 220 may learn the neural network by tuning parameters for a plurality of layers of the neural network using a backpropagation algorithm.

Meanwhile, the learning module 220 may derive an optimal parameter for the neural network by repeatedly performing the above-described learning steps.

In other words, the learning module 220 may determine whether the preset termination conditions are satisfied S95.

For example, the user may set to repeat the learning process until the loss function of the neural network does not decrease.

Here, the loss function may mean a degree of similarity between correct answer data for input data and output data of the neural network.

The loss function is used to guide the learning process of the neural network. For example, a mean square error (MSE), a cross entropy error (CEE), etc. may be used.

Alternatively, the user may set, for example, to repeat the learning process for a predetermined number of times.

For example, when it is determined that the preset termination conditions are not satisfied, the learning module 220 may return to S101 to repeat the learning process.

In contrast, when it is determined that the preset termination conditions are satisfied, the learning module 220 may terminate the learning process.

Therefore, according to the learning method according to one exemplary embodiment of the present disclosure, it is possible to learn an optimal classification criterion for identifying types of cells in a cell image, and the image analysis module may accurately identify the types of cells using the pre-learned classification criterion.

In other words, according to the image analysis method according to exemplary embodiments of the present disclosure, the types of cells may be automatically identified from the unstained blood cell image, and thus it may be possible to more accurately and rapidly provide blood analysis results.

### 6. Fourth Exemplary Embodiment

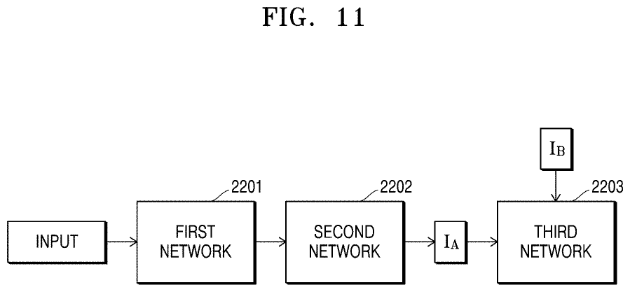

FIG. 11 is a diagram illustrating an image synthesis method for converting an unstained blood cell image into a stained blood cell image according to a fourth exemplary embodiment of the present disclosure.

The learning process according to the fourth exemplary embodiment of the present disclosure may be performed in the above-described learning module 220, and may be performed using at least one neural network.

For example, the neural network may include a plurality of networks, and may include at least one convolutional neural network and deconvolutional neural network.

In addition, input data (Input) applied to the neural network may be training data generated through the above-described training data construction module 210. The training data may be an unstained blood cell image, and may be data in which label information regarding the types of cells in the blood cell image is matched.

For example, when the unstained blood cell image is input to a first network 2201, features regarding the user's ROI (e.g., neutrophils, eosinophils, basophils, lymphocytes, monocytes, etc.) in the unstained blood cell image may be extracted. The process of extracting features in the input data from the first network 2201 may correspond to an operation performed by the above-described learning module 220.

Next, a second network 2202 may synthesize the unstained blood cell image (Input) into a stained blood cell image (IA) using a plurality of features extracted through the above-described first network 2201.

In addition, a third network 2203 may receive the stained blood cell image (IA) synthesized through the second network 2202 and an actual stained cell image (IB). In this regard, the third network may calculate the degree of similarity between the synthesized stained blood cell image and the actual stained cell image (IB).

Meanwhile, the second network 2202 and the third network 2203 may be trained to allow the above-described second network to synthesize an image close to the actual stained cell image. For example, the learning process may be repeatedly performed until the similarity value calculated by the third network exceeds a preset level. In this regard, the learning process using the neural network may be performed in a manner similar to the learning methods described through the first to third exemplary embodiments.

Therefore, according to the learning method according to the fourth exemplary embodiment of the present disclosure, even when a user inputs an unstained blood cell image, it is possible to provide a stained blood cell image by performing learning to convert the unstained blood cell image into the stained blood cell image. Therefore, the user may intuitively recognize the types of cells in the blood cell image without staining.

The above-described methods according to exemplary embodiments may be implemented in the form of executable program command through various computer means recordable to computer-readable media. The computer-readable media may include, alone or in combination, program commands, data files, data structures, etc. The program commands recorded to the media may be components specially designed for the exemplary embodiment or may be usable to a skilled person in the field of computer software. Examples of the computer readable record media include magnetic media such as hard disk, floppy disk, magnetic tape, optical media such as CD-ROM and DVD, magneto-optical media such as floptical disk, and hardware devices such as ROM, RAM and flash memory specially designed to store and carry out programs. Examples of the program commands include not only a machine language code made by a complier but also a high level code that may be used by an interpreter etc., which is executed by a computer. The above-described hardware device may be configured to work as one or more software modules to perform the action of the exemplary embodiment and they may do the same in the opposite case.

As described above, although the exemplary embodiments have been described by the limited embodiments and drawings, various modifications and variations are possible from the above descriptions by those skilled in the art. For example, adequate results may be achieved even if the foregoing techniques are carried out in different order than described above, and/or the aforementioned elements, such as systems, structures, devices, or circuits, are combined or coupled in different forms from those as described above or are substituted or switched with other components or equivalents.

Thus, other implementations, alternative embodiments, and equivalents to the claimed subject matter are construed as being within the appended claims.

### REFERENCE NUMERALS

100: Image capture device
200: Computing device
210: Training data construction module
220: Learning module
230: Image analysis module
300: User device

1. An image analysis method comprising:
obtaining an unstained cell image;
obtaining at least one feature map comprised in the cell image; and
identifying a type of cell corresponding to the feature map by using a preset criterion.

2. The image analysis method of claim 1, wherein the preset criterion is a criterion pre-learned to classify the type of cell comprised in the unstained cell image.

3. The image analysis method of claim 1, wherein the preset criterion is learned using training data obtained by matching label information of a reference image after staining with a target image before staining.

4. The image analysis method of claim 2, wherein the preset criterion is continuously updated to accurately identify the type of cell from the unstained cell image.

5. The image analysis method of claim 3, wherein the matching of the label information comprises
extracting one or more features from the target image and the reference image;
matching features of the target image and the reference image; and
transmitting label information comprised in the reference image to a pixel corresponding to the target image.

6. The image analysis method of claim 1, further comprising segmenting the unstained cell image, based on an user's region of interest, before the obtaining of the feature map.

7. The image analysis method of claim 6, wherein the type of cell is identified according to the preset criterion for each region of the segmented image.

8. The image analysis method of claim 1, wherein the number of each type of the identified cell is counted and further provided.

9. The image analysis method of claim 1, further providing a diagnosis result regarding a specific disease, based on information of the identified cell type.

10. A learning method for analyzing a blood image using at least one network, the learning method comprising:
obtaining one or more training data of unstained blood;
generating at least one feature map from the training data;
outputting prediction data of the feature map, based on one or more predefined categories; and
tuning a parameter applied to the network, based on the prediction data,
wherein the above-described steps are repeatedly performed until preset termination conditions are satisfied.

11. The learning method of claim 10, wherein the training data comprises label information regarding one or more cells comprised in the blood.

12. The learning method of claim 11, wherein the label information is obtained by matching label information of reference data after staining with unstained target data.

13. The learning method of claim 10, wherein the training data is data segmented according to the preset criterion.

14. The learning method of claim 10, wherein the training data is applied as a plurality of segments according to an user's region of interest.

15. The learning method of claim 10, wherein, when it is determined that the preset termination conditions are satisfied, learning is terminated.

16. A computer-readable medium having recorded thereon a program for executing the method of claim 1 on a computer.
